(19) United States
(12) Patent Application Publication
(10) Pub. No.: US 2025/0249265 A1
(43) Pub. Date: Aug. 7, 2025

(54) SYSTEMS AND METHODS FOR AUTOMATED FITTING OF MULTI-ELECTRODE CUFFS

(71) Applicant: The Alfred E. Mann Foundation for Scientific Research, Valencia, CA (US)

(72) Inventors: Brian SHELTON, San Buenaventura, CA (US); Hitesh Shyamkumar HOTLANI, Santa Clarita, CA (US)

(21) Appl. No.: 19/047,520

(22) Filed: Feb. 6, 2025

Related U.S. Application Data
(60) Provisional application No. 63/550,276, filed on Feb. 6, 2024.

(51) Int. Cl. A61N 1/372; A61N 1/36

(52) U.S. Cl. CPC A61N 1/37241; A61N 1/3615; A61N 1/36175; A61N 1/36185; A61N 1/37217; A61N 1/37247; A61N 1/37264

(57) ABSTRACT

The present disclosure provides systems and methods that utilize automation to expedite the fitting process for implantable multiple-electrode peripheral neural stimulators.

### CROSS-REFERENCE TO RELATED APPLICATION

The present application claims the benefit of priority to U.S. Provisional Application No. 63/550,276, filed Feb. 6, 2024, the entire contents of which is incorporated by reference in its entirety.

### TECHNICAL FIELD

The present disclosure relates to systems and methods that utilize automation to expedite the fitting process for implantable multiple-electrode peripheral neural stimulators.

### BACKGROUND

Implantable stimulation devices, such as implantable pulse generators (“IPGs”) are often used in the context of neuromodulation therapy, and, in general, comprise a hermetically sealed housing containing stimulation circuitry. Electrically conductive feedthrough pins extend from the stimulation circuitry and into a header connector, which is mounted upon the IPG housing, and the header connector defines a receptacle adapted to receive and couple with the proximal end of a stimulation lead, which includes an elongate lead body and a nerve cuff. For direct stimulation of a nerve, the nerve cuff electrodes may be configured to be secured directly to, wrapped around, or positioned next to the target nerve(s). Stimulation leads may be manufactured in a variety of configurations to accommodate different applications or anatomical targets. For example, a non-exhaustive list of nerve cuffs includes those illustrated and/or described in U.S. Pat. Nos. 10,967,178, 11,844,941, and 11,951,301, as well as in U.S. Pre-Grant Pub. Nos. 2024/0082572A1, 2024/0058602A1, and 2024/0108883A1, which are each incorporated herein by reference in their entirety.

In recent years, IPGs have received attention as a potential solution for treating or mitigating obstructive sleep apnea (“OSA”). OSA is a highly prevalent sleep disorder that is caused by the collapse of or increase in the resistance of the pharyngeal airway, often resulting from tongue obstruction caused by reduced genioglossus muscle activity during the deeper states of non-rapid eye movement (NREM) sleep. Obstruction of the upper airway causes breathing to pause during sleep. Cessation of breathing, in turn, causes a decrease in the blood oxygen saturation level, which is eventually corrected when the person wakes up and resumes breathing. The long-term effects of OSA include, but are not limited to, high blood pressure, heart failure, strokes, diabetes, headaches, and general daytime sleepiness and memory loss. An IPG may be implanted into a patient, and used to stimulate one or more branches of the hypoglossal nerve which innervates the upper airway muscle, to increase the patency of the upper airway of the patient, thereby preventing or minimizing the onset of OSA.

IPGs intended for treating OSA are typically implanted in the upper chest of a patient. During the same procedure, a surgeon may also make a small incision in the skin under the jaw in order to implant and fit one or more electrode cuffs around branches of the hypoglossal nerve. It is desirable that any electrode implanted in contact with a nerve, such as the hypoglossal nerve, continually remain in firm contact with such nerve to maximize the effectiveness of the stimulation regimen. After positioning the IPG, a surgeon will then normally connect one or more leads from the electrodes to the IPG. The connection is then tested. If the IPG is properly connected and working, the patient's tongue should stick out when current is applied to the hypoglossal nerve via the implanted electrodes.

In the case of multi-electrode stimulation devices (e.g., comprising a plurality of electrodes associated with the lead(s) or nerve cuff(s), the number of permutations to explore increases exponentially. As such, the initial testing process for such devices is often time consuming, delaying the completion of surgery. This delay increases the risk of surgical complications and wastes hospital resources. Moreover, the added complexity of multi-electrode configurations also complicates follow-up visits after surgery, as a clinician programmer seeking to optimize treatment would have to test out numerous potential combinations of electrodes and parameters thereof.

### SUMMARY OF SELECTED ASPECTS

The devices, systems, and methods described herein address various shortcomings in the art, e.g., by automating the fitting process for neurostimulation systems, allow for more efficient use of medical resources and reducing the possibility of complications arising from prolonged surgery. The methods described herein may also be used to adjust or optimize one or more of the stimulation parameters used by an implanted neurostimulation system following the initial surgical implantation.

In a first general aspect, the disclosure provides a neurostimulation system, comprising: an implantable stimulator configured to deliver electrical stimulation to at least one nerve of a human subject via a plurality of electrodes or electrode pairs, and an implantable controller configured to control one or more parameters of the delivery of the electrical stimulation to the at least one nerve of the human subject, wherein the controller is configured to operate in a first fitting mode, based on user input wirelessly communicated to the controller, the first fitting mode comprising a mode wherein the controller is configured to: a) deliver stimulation sequentially, for a predetermined stimulation duration and at a predetermined pulse amplitude, to each of at least a subset of the plurality of electrodes or electrode pairs, b) repeat step a) one or more times, gradually increasing a pulse amplitude of stimulation during each repeat cycle, starting from the predetermined pulse amplitude, c) cease delivering stimulation, when a predetermined maximum pulse amplitude is reached or upon receipt of user input instructing the controller to cease stimulation, and d) store a pulse amplitude parameter for one or more of the electrodes or electrode pairs used in steps a)-c), and optionally a selection of one or more of the electrodes or electrode pairs used in steps a)-c), provided via user input, in a memory of the controller, wherein the stored parameter is indicative of a minimum threshold for stimulation.

In some aspects, the controller is further configured to operate in a second fitting mode, based on user input wirelessly communicated to the controller from an electronic device, the second fitting mode comprising a mode wherein the controller is configured to: a) deliver stimulation to at least one electrode or electrode pair selected based on user input, for a predetermined stimulation duration and at a predetermined pulse amplitude, b) gradually increase a pulse amplitude of stimulation delivered to the electrode or electrode pair selected in step a), starting from the predetermined pulse amplitude, c) stop delivering stimulation, when a predetermined maximum pulse amplitude is reached or upon receipt of user input instructing the controller to cease stimulation, and d) store a pulse amplitude parameter for one or more of the electrodes or electrode pairs used in steps a)-c) in a memory of the controller, wherein the stored parameter is indicative of a maximum threshold for stimulation.

In some aspects, the controller is further configured to operate in a third fitting mode, based on user input wirelessly communicated to the controller from an electronic device, the third fitting mode comprising a mode wherein the controller is configured to: a) receive minimum and maximum pulse amplitude parameters for at least one electrode or electrode pair in the plurality of electrodes or electrode pairs, based on user input wirelessly communicated to the controller from the electronic device, and to store the received minimum and maximum parameters in a memory of the controller.

In some aspects, the electronic device comprises a dedicated controller of the neurostimulation system, or a smartphone, tablet, or computer configured to execute a software application that provides communication with and control of the controller.

In some aspects, the predetermined stimulation duration used in one or more of the fitting modes, comprises a) 0.5, 1.0, 1.5, 2.0, 2.5, 3.0, 3.5, 4.0, 4.5, or 5.0 seconds, or b) a duration within a range defined by a pair of endpoints selected from any of the lengths of time listed in a).

In some aspects, the predetermined pulse amplitude used in one or more of the fitting modes, comprises a) 0.01, 0.02, 0.03, 0.04, 0.05, 0.06, m 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, 0.25, 0.30, 0.35, 0.40, 0.45, 0.50, 0.60, 0.70, 0.80, 0.90, 1.00, 1.10, 1.20, 1.30, 1.40, 1.50, 1.60, 1.70, 1.80, 1.90, 2.00, 2.50, 3.00, 3.50, 4.00, 4.50, 5.00, 5.50, or 6.00 mA, or b) a pulse amplitude within a range defined by a pair of endpoints selected from any of the amounts listed in a).

In some aspects, the controller is configured to gradually increase the pulse amplitude delivered in step b), in one or more of the fitting modes, by incrementing the pulse amplitude by i) 0.01, 0.02, 0.03, 0.04, 0.05, 0.06, 0.07, 0.08, 0.09, or 0.10, 0.11, 0.12, 0.13, 0.14, 0.15, 0.16, 0.17, 0.18, 0.19, 0.20, 0.21, 0.22, 0.23, 0.24, 0.25, 0.30, 0.35, 0.40, 0.45, 0.50, 0.60, 0.70, 0.80, 0.90, 1.00, 1.10, 1.20, 1.30, 1.40, 1.50, 1.60, 1.70, 1.80, 1.90, 2.00, 2.50, 3.00, 3.50, 4.00, 4.50, 5.00, 5.50, or 6.00 mA each time, or ii) by incrementing the pulse amplitude by an amount within a range defined by a pair of endpoints selected from any of the amounts listed in i).

In some aspects, the controller is configured to operate in the first fitting mode, the second fitting mode, and the third fitting mode, and alternate between, activate, or deactivate, a current fitting mode, based on user input received from the electronic device.

In a second general aspect, the disclosure provides a method for fitting a human subject with a neurostimulation system, comprising: providing an implantable stimulator configured to deliver electrical stimulation to at least one nerve of a human subject via a plurality of electrodes or electrode pairs, providing an implantable controller configured to control one or more parameters of the delivery of the electrical stimulation to the at least one nerve of the human subject; receiving, by an electronic device configured to wirelessly communicate with and control one or more parameters of the controller, information identifying one or more covered and/or uncovered electrodes or electrode pairs in the plurality of electrodes or electrode pairs; transmitting, by the electronic device, an instruction to the controller to initiate a first fitting mode, the first fitting mode comprising a mode wherein the controller is configured to: a) deliver stimulation sequentially, for a predetermined stimulation duration and at a predetermined pulse amplitude, to each of the uncovered electrodes or electrode pairs, b) repeat step a) one or more times, gradually increasing a pulse amplitude of stimulation during each repeat cycle, starting from the predetermined pulse amplitude, c) cease delivering stimulation, when a predetermined maximum pulse amplitude is reached or upon receipt of user input instructing the controller to cease stimulation, and d) store a pulse amplitude parameter for one or more of the uncovered electrodes or electrode pairs used in steps a)-c), wherein the stored parameter is indicative of a minimum threshold for stimulation.

In some aspects of the methods described herein, the controller is further configured to operate in a second fitting mode, the second fitting mode comprising a mode wherein the controller is configured to: a) deliver stimulation to at least one electrode or electrode pair selected based on user input, for a predetermined stimulation duration and at a predetermined pulse amplitude, b) gradually increase a pulse amplitude of stimulation delivered to the electrode or electrode pair selected in step a), starting from the predetermined pulse amplitude, c) stop delivering stimulation, when a predetermined maximum pulse amplitude is reached or upon receipt of user input instructing the controller to cease stimulation, and d) store a pulse amplitude parameter for one or more of the electrodes or electrode pairs used in steps a)-c), wherein the stored parameter is indicative of a maximum threshold for stimulation; and the method further comprises transmitting, by the electronic device, an instruction to the controller to initiate the second fitting mode.

In some aspects of the methods described herein, the controller is further configured to operate in a third fitting mode, the third fitting mode comprising a mode wherein the controller is configured to: a) receive minimum and maximum pulse amplitude parameters for at least one electrode or electrode pair in the plurality of electrodes or electrode pairs; and the method further comprises transmitting, by the electronic device, an instruction to the controller to initiate the third fitting mode.

In some aspects of the methods described herein, the electronic device comprises a dedicated controller of the neurostimulation system, or a smartphone, tablet, or computer configured to execute a software application that provides communication with and control of the controller.

In some aspects of the methods described herein, the predetermined pulse amplitude used in one or more of the fitting modes, comprises a) 0.5, 1.0, 1.5, 2.0, 2.5, 3.0, 3.5, 4.0, 4.5, or 5.0 seconds, or b) a duration within a range defined by a pair of endpoints selected from any of the lengths of time listed in a).

In some aspects of the methods described herein, the predetermined pulse amplitude used in one or more of the fitting modes, comprises a) 0.01, 0.02, 0.03, 0.04, 0.05, 0.06, m 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, 0.25, 0.30, 0.35, 0.40, 0.45, 0.50, 0.60, 0.70, 0.80, 0.90, 1.00, 1.10, 1.20, 1.30, 1.40, 1.50, 1.60, 1.70, 1.80, 1.90, 2.00, 2.50, 3.00, 3.50, 4.00, 4.50, 5.00, 5.50, or 6.00 mA, or b) a pulse amplitude within a range defined by a pair of endpoints selected from any of the amounts listed in a).

In some aspects of the methods described herein, the controller is configured to gradually increase the pulse amplitude delivered in step b), in one or more of the fitting modes, by incrementing the pulse amplitude by i) 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, 0.25, 0.30, 0.35, 0.40, 0.45, 0.50, 0.60, 0.70, 0.80, 0.90, 1.00, 1.10, 1.20, 1.30, 1.40, 1.50, 1.60, 1.70, 1.80, 1.90, 2.00, 2.50, 3.00, 3.50, 4.00, 4.50, 5.00, 5.50, or 6.00 mA each time, or ii) by incrementing the pulse amplitude by an amount within a range defined by a pair of endpoints selected from any of the amounts listed in i).

In some aspects of the methods described herein, the method further comprises transmitting, by the electronic device to the controller, instructions to initiate the first fitting mode, followed by instructions to enter the second fitting mode, and/or the third fitting mode.

In some aspects of the methods described herein, the controller is further configured to pause between any or all of steps a)-d) of the first fitting mode and/or the second fitting mode, until an instruction to proceed is received from the electronic device.

In some aspects of the methods described herein, the instruction transmitted by the electronic device comprises user input provided by a clinician programmer.

In some aspects of the methods described herein, the electronic device is a dedicated controller comprising a portable electronic device configured to wirelessly communicate with and control a plurality of neurostimulation systems.

In still further aspects, disclosure provides methods of treating OSA using any of the systems described herein, e.g. following the optimization of one or more stimulation parameters using a fitting method according to the present disclosure.

In third general aspect, the disclosure provides method for adjusting or optimizing a neurostimulation system, comprising: providing an implantable stimulator configured to deliver electrical stimulation to at least one nerve of a human subject via a plurality of electrodes or electrode pairs; providing an implantable controller configured to control one or more parameters of the delivery of the electrical stimulation to the at least one nerve of the human subject; receiving, by an electronic device configured to wirelessly communicate with and control one or more parameters of the controller, information identifying one or more electrodes or electrode pairs in the plurality of electrodes or electrode pairs; transmitting, by the electronic device, an instruction to the controller to initiate a first fitting mode, the first fitting mode comprising a mode wherein the controller is configured to: a) deliver stimulation sequentially, for a predetermined stimulation duration and at a predetermined pulse amplitude, to one or more electrodes or electrode pairs positioned adjacent to at least one of the identified electrodes or electrode pairs, b) repeat step a) one or more times, gradually increasing a pulse amplitude of stimulation during each repeat cycle, starting from the predetermined pulse amplitude, c) cease delivering stimulation, when a predetermined maximum pulse amplitude is reached or upon receipt of user input instructing the controller to cease stimulation, and d) store a pulse amplitude parameter for one or more of the electrodes or electrode pairs used in steps a)-c), and optionally a selection of one or more of the electrodes or electrode pairs used in steps a)-c), received from the electronic device, wherein the stored parameter is indicative of a minimum threshold for stimulation. Such methods may be used, e.g., to identify preferred or optimal electrode(s) for stimulation after an electrode cuff has migrated post-implantation.

### BRIEF DESCRIPTION OF THE DRAWINGS

FIG. 1 is a block diagram illustrating an exemplary embodiment of a neurostimulation system compatible with the automated fitting methods described herein.

FIG. 2 is a conceptual flow diagram of a process for automated fitting of a neurostimulation system according to one non-limiting aspect of the disclosure.

FIG. 3 is a conceptual flow diagram of another process for automated fitting of a neurostimulation system according to one non-limiting aspect of the disclosure.

FIG. 4 is a is a block diagram of various system components, capable of being used in an electronic device (e.g., a computer or dedicated controller) configured to implement or perform one or more of the functions described herein.

The exemplary systems and methods shown in the foregoing drawings represent non-limiting examples and do not constrain the scope of the inventions described herein, which are defined solely by the claims.

### DETAILED DESCRIPTION

The detailed description set forth below in connection with the appended drawings is intended as a description of various configurations and is not intended to represent the only configurations in which the concepts described herein may be practiced. The detailed description includes specific details for the purpose of providing a thorough understanding of various concepts. However, it will be apparent to those skilled in the art that these concepts may be practiced without these specific details. In some instances, well known structures and components are shown in block diagram form in order to avoid obscuring such concepts.

Several aspects of exemplary embodiments according to the present disclosure will now be presented with reference to various systems and methods. These systems and methods will be described in the following detailed description and illustrated in the accompanying drawings by various blocks, components, circuits, processes, algorithms, etc. (collectively referred to as “elements”). These elements may be implemented using electronic hardware, computer software, or any combination thereof. Whether such elements are implemented as hardware or software depends upon the particular application and design constraints imposed on the overall system.

By way of example, an element, or any portion of an element, or any combination of elements may be implemented as a “processing system” or “controller” that includes one or more processors. Examples of processors include microprocessors, microcontrollers, graphics processing units (“GPUs”), central processing units (“CPUs”), application processors, digital signal processors (“DSPs”), reduced instruction set computing (“RISC”) processors, systems on a chip (“SoC”), baseband processors, field programmable gate arrays (“FPGAs”), programmable logic devices (“PLDs”), application-specific integrated circuits (“ASICs”), state machines, gated logic, discrete hardware circuits, and other suitable hardware configured to perform the various functionality described throughout this disclosure. One or more processors in the processing system may execute software. Software shall be construed broadly to mean instructions, instruction sets, code, code segments, program code, programs, subprograms, software components, applications, software applications, software packages, routines, subroutines, objects, executables, threads of execution, procedures, functions, etc., whether referred to as software, firmware, middleware, microcode, hardware description language, or otherwise.

Accordingly, in one or more exemplary embodiments, the functions described may be implemented in hardware, software, or any combination thereof. If implemented in software, the functions may be stored on or encoded as one or more instructions or code on a computer-readable medium. Computer-readable media includes computer storage media. Storage media may be any available media that can be accessed by a computer. By way of example, and not limitation, such computer-readable media can comprise a random-access memory (“RAM”), a read-only memory (“ROM”), an electrically erasable programmable ROM (“EEPROM”), optical disk storage, magnetic disk storage, other magnetic storage devices, combinations of the aforementioned types of computer-readable media, or any other medium that can be used to store computer executable code in the form of instructions or data structures that can be accessed by a computer.

The present disclosure provides, inter alia, systems and methods intended to expedite the fitting process for implantable multiple-electrode peripheral neural stimulators. These systems and methods can also be used to expedite the initial (or a follow-up) clinical visit after implantation of a neurostimulation system, by allowing a clinical programmer to quickly identify the optimum stimulation parameters for a given patient. Automation of the other time-consuming fitting and optimization workflows provides various potential benefits, including improved patient care, more efficient use of limited medical resources (e.g., physician time), and a reduction in the likelihood of surgical complications due to prolonged surgery.

As explained above, neurostimulation systems typically include an IPG and one or more stimulation leads terminating in electrodes placed on or in proximity to nerve targeted for stimulation. For example, nerve cuffs may be placed on or around one or more branches of a target nerve (or nerves) in order to deliver electrical stimulation and to avoid displacement of the electrodes post-surgery. During the initial surgical implantation of a neurostimulation system, testing is normally performed to verify that each nerve cuff is on the correct nerve and operating as intended. For example, a clinician programmer may have a surgical assistant test stimulation and specify the number of electrodes in contact with each target nerve, the size of each target nerve, and/or the outer diameter of the nerve cuff. Using this information, the clinician programmer may determine which electrodes are in contact with the target nerve and deactivate the remaining electrodes.

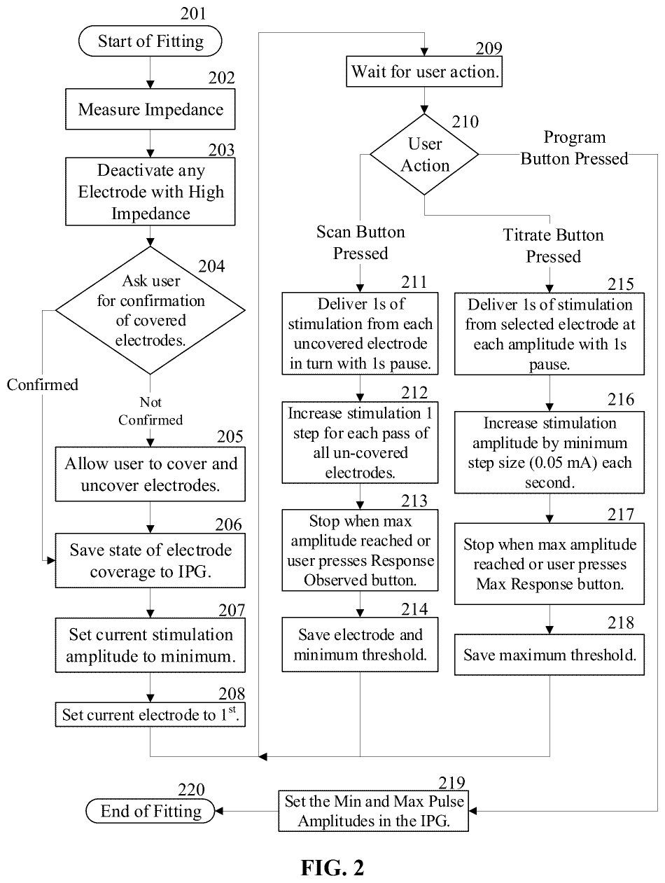

The present systems and methods may be used to automate the fitting process, e.g., by providing a computer-implemented system for efficiently determining minimum and maximum pulse amplitude parameters for a plurality of electrodes or electrode pairs. For example, in a general exemplary aspect, a fitting session may begin with the clinician programmer pairing an electronic device (e.g., a dedicated control unit) wirelessly with the implanted controller of the neurostimulation system. Once a connection is established, the clinician programmer may be presented with the option to place the controller in one or more fitting modes (e.g., as illustrated by FIG. 2).

In a first fitting mode, initiated by the clinician programmer via an interface of the electronic device, the controller may be configured to cause the IPG to deliver a short stimulation burst sequentially using each active electrode. When a cycle is complete, the controller may be configured to pause (e.g., allowing a clinician programmer to command the IPG to increase the stimulation amplitude and to proceed through another cycle of sequential stimulation). Alternatively, the controller may be configured to automatically increase the stimulation amplitude and begin another cycle until a command to pause or terminate the testing process is received from the clinician programmer, or until a predetermined maximum amplitude is reached. In typical operation, it is envisioned that the clinician programmer will either actively initiate repeat cycles as needed, or allow for automatic operation, while monitoring the subject for a desired response to the stimulation. At that time, the clinician programmer may terminate or pause stimulation.

It is further envisioned that the interface of the electronic device may be configured to allow the clinician programmer to easily select and/or change the electrode or electrode pairs being used to deliver stimulation, allowing for the clinician programmer to quickly move on to testing alternative or additional electrodes (or electrode pairs), in turn. In this manner, the clinician programmer can quickly identify the optimal electrode(s) or pair(s)/combination(s) thereof, to stimulate. Typically, the goal of the clinician programmer would be to use the selected pulse amplitude as a minimum amplitude for stimulation.

After scanning for the correct electrode(s) (or pair(s)/combination(s) thereof), the clinician programmer may then use the interface of the electronic device to instruct the controller to enter a second fitting mode, e.g., one designed to identify the maximum stimulation amplitude above which the stimulation response does not change. The maximum amplitude for each of the selected electrode(s) (or pair(s)/combination(s) thereof) may be determined, e.g., using a titration process wherein the stimulation amplitude is gradually increased by a predetermined amount. Again, the clinician programmer may monitor the patient's response in order to identify the threshold wherein the response does not change. In some aspects, the gradual titration process may be used to identify an approximate maximum pulse amplitude and the interface of the electronic device may allow the clinician programmer to stop the titration in order to manually enter or adjust the amplitude of stimulation being delivered. For example, the interface may allow the clinician programmer to stop titration and to increase or decrease the pulse amplitude and deliver a short stimulation burst. When the clinician programmer determines that they have found the maximum amplitude for a given electrode (or a pair or combination of electrodes), they may be set the controller to utilize these values for the desired electrode(s) or any combination(s) or pair(s) thereof.

In some cases, stimulation cannot begin on the day of implantation (e.g., due to scheduling issues or surgical complications), and an initial clinical visit may take place days or weeks post-implantation (e.g., 4-8 weeks later). During the recovery period following surgery, in some cases nerve cuffs may move and/or impedance may change due to tissue growth around the nerve cuff. As such, it is envisioned that the automated systems and methods described herein may also be used to adjust or optimize stimulation parameters post-implantation.

In addition to expediting the fitting process using automated testing as described above (and elsewhere herein), it is envisioned that the interface of the electronic device may be further configured to help expedite the fitting process. For example, the interface may be configured to allow a clinician programmer to select one or more desired electrodes, or combinations/pairs thereof, and to (a) allow the clinician programmer to specify that stimulation should also be tested using (a) one or more adjacent electrodes, or combinations/pairs thereof, or (b) electrodes, or combinations/pairs thereof within a particular distance of a selected electrode or combination/pair thereof. For example, the interface may allow a clinician programmer to select an electrode, and to initiate stimulation, or the titration of stimulation, using all (or any subset) of the electrodes adjacent to the selected electrode, or any or all electrodes within a given distance of the selected electrode. Without being bound to a theory, it is believed that such methods may allow for rapid identification of optimal electrodes (or combinations/pairs thereof) and stimulation amplitude parameters, for a given patient.

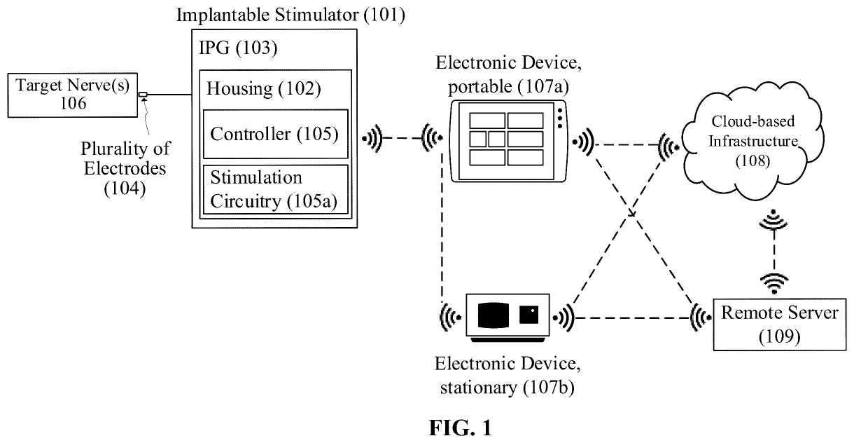

FIG. 1 is a block diagram of an exemplary neurostimulation system according to the present disclosure, which would be compatible with the automated methods described herein. This example illustrates an implantable neurostimulator (101), in the form of an IPG (103) comprising a hermetically sealed housing (102); a controller (105) within the housing (102), comprising a processor and memory, and configured to control one or more settings or functions of the IPG; stimulation circuitry (105a) within the housing (102), configured to provide electrical stimulation pulses to one or more of a plurality of electrodes (104) positioned at a distal end of one or more stimulation leads extending from the IPG (103), for stimulating at least one target nerve (106) of the subject. The implantable stimulator (101) is shown to be capable of wireless communication with a portable electronic device (107a). The portable electronic device may comprise, e.g., a dedicated controller, or a smartphone, or other portable electronic device configured to execute one or more software applications or executable code that allows the device to interact with and/or control one or more parameters of the controller (105) of the implantable neurostimulator (101). A stationary electronic device (107b) is also shown as another potential component of the system. Stationary devices are envisioned as devices that are typically operated while stationary (e.g., a desktop computer), and which are configured to execute one or more software applications or executable code configured to interact with and/or control one or more parameters of the controller (105) of the implantable neurostimulator (101). In this case, the controller (105) is shown to wirelessly communicate with the portable electronic device (107a). However, in other aspects it may communicate with a stationary electronic device (107b) or with any of the other potential components of systems described herein.

In some aspects, the portable electronic device (107a) or the stationary electronic device (107b) may be configured to communicate with one or more implantable or external sensors. For example, the portable electronic device (107a) or the stationary electronic device (107b) may be capable of obtaining data from one or more implanted or external electromyogram (EMG) sensors configured to detect a signal indicative of the subject's response to stimulation delivered by the IPG, allowing the device to display, monitor, and/or log the subject's response to stimulation (e.g., during the testing or titration processes described herein). While an EMG sensor is noted as an example, it is envisioned that any other implantable or external sensor(s) capable of detecting a signal indicative of a biometric parameter of the subject may alternatively be used in various aspects of the systems described herein. FIG. 1 further illustrates the potential use of a remote server (109) and cloud-based infrastructure (108). Remote servers (109) may be used, e.g., to store logs comprising sensor and/or stimulation-related data (e.g., to allow a medical practitioner or clinician programmer to review such data from a remote location or at a later time). In some aspects, a remote server (109) may be configured to set or modify one or more parameters of the controller (105) of the implantable stimulator (101). For example, a remote medical practitioner or clinician programmer may be allowed to review sensor and/or stimulation-related data for the subject (e.g., stored on the remote server 109) and to adjust one or more thresholds or other parameters related to treatment. In some aspects, the remote server (109) may also serve as a repository for sensor data collected by third-party devices (e.g., sensors integrated into a user's smartphone or smartwatch, etc.), that is made accessible to the controller (105) or to any communicatively-linked devices such as the portable or stationary electronic devices (107a, 107b) shown in this example. Such data may be used by the systems described herein, e.g., to set or modify parameters for treatment.

FIG. 2 is flowchart illustrating an exemplary method for automating the fitting of a subject with a neurostimulation system according to the present disclosure. In this example, the fitting process begins during the surgical implantation of a neurostimulation system (e.g., comprising an implantable controller and an IPG as described herein). In this example, the fitting process begins (step 201) with a clinician programmer (or an assistant thereof) may measure the impedance of one or more of the electrodes (or any combinations/pairs thereof) (step 202). Any electrodes found to have an impedance above a given threshold may optionally be deactivated at this stage (Step 203). Next, the system may present the user (e.g., a clinician programmer) with a request to identify or confirm which electrodes are covered (step 204). As shown by this flowchart, the system may be configured to allow a user to cover/uncover electrodes (Step 205) at this stage, and to save the state of electrode coverage following confirmation (Step 206). A covered electrode refers to a configuration where the cuff wraps around the nerve and has sufficient overlap such that at least one of the electrodes lies on top of the cuff and not the nerve.

Next, the system may be configured to set the stimulation amplitude to a minimum value. However, it is envisioned that in other embodiments a minimum stimulation amplitude may be set prior to this process (e.g., a default value may be preprogrammed). In either case, at step 207 the system may be configured to allow a user to confirm, select, or enter a minimum stimulation amplitude for testing. At this stage, at least one electrode, or a pair or combination thereof, may be selected as a first electrode (or pair/combination thereof) for testing. As illustrated by this flowchart, the systems described herein may allow a user to select one or more fitting modes at this stage (step 209). A clinician programmer may, e.g., select a scanning mode, a titration mode, or a program mode, based on buttons or other elements provided by the graphical user interface (GUI) or input system provided by the electronic device (e.g., the portable electronic device (107a) or the stationary electronic device (107b) shown in FIG. 1).

For example, a clinician programmer may select a first fitting mode wherein stimulation is delivered to the selected electrode(s) (or combination/pair thereof), optionally for a predetermined amount of time. Here, stimulation is applied for one second to each uncovered electrode, with a one-second pause (step 211). Next, stimulation is increased from the minimum stimulation amplitude by a predetermined increment (e.g., 0.05 mA) and then delivered to all uncovered electrodes (step 212). This process is then repeated one or more times until the user enters a command to pause or terminate the process (e.g., upon detecting that response to stimulation has been observed), or until a predetermined maximum stimulation amplitude is reached (step 213). It is envisioned that the system may be set to automatically increase and repeat stimulation during this step, or to pause after each cycle in order to wait for user confirmation to proceed. In either case, the minimum stimulation amplitude threshold may be recorded for each tested electrode (or pair/combination thereof) (step 214).

At this stage, the interface of the electronic device may allow a user to select an alternative fitting mode (returning to steps 209 and 210). Alternatively, the system may be configured to automatically proceed to a second fitting mode. In either case, as shown here, the system may provide a second fitting mode wherein stimulation is delivered to each selected electrode (or pair/combination thereof), optionally for a predetermined time (step 215). The initial stimulation amplitude delivered in this second mode may comprise a predetermined stimulation amplitude (e.g., manually entered by the user or provided as a default value). Typically, the initial stimulation will be equal to or greater than the minimum stimulation amplitude recorded during the first fitting mode. In any event, the initial stimulation amplitude may be gradually titrated upwards (step 216) until a maximum amplitude is reached or when a user enters an instruction to pause or stop stimulation (step 217). In this example, stimulation is titrated based on a fixed increment of 0.05 mA each cycle, however in other embodiments an alternative fixed value may be used (e.g., 0.01, 0.02, 0.03, 0.04, 0.05, 0.06, 0.07, 0.08, 0.09, or 0.10 mA per cycle). Alternatively, the titration may be performed with a variable increment (e.g., the increment may gradually decrease over time or in response to the user selecting a rate of change for the increment, or an option to fine-tune the stimulation whereby a smaller increment is used). As noted above, a user (e.g., the clinician programmer) may pause or terminate stimulation when it is determined that increasing the amplitude of stimulation provides no further effect, or for any other reason. This fitting mode concludes in the illustrated example with the maximum amplitude being recorded for each of the tested electrodes, or combinations/pairs thereof.

As illustrated by this figure, the interface of the system may provide a third fitting mode allowing the user to manually enter minimum and/or maximum stimulation amplitudes for one or more electrodes or combinations/pairs thereof, or to set the recorded minimum and/or maximum amplitude(s) determined in the first and second fitting modes, as active parameters for treatment (step 219). This third mode thus illustrates the adoption of personalized stimulation parameters for treatment for a given patient.

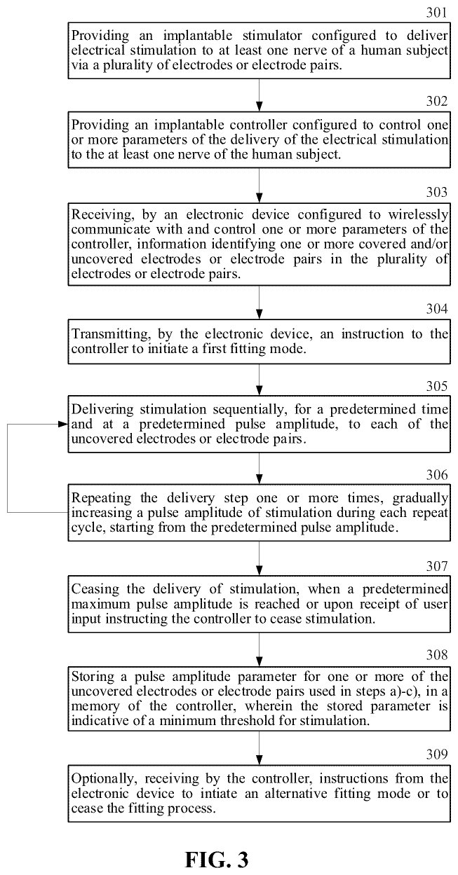

FIG. 3 illustrates another exemplary workflow for automated fitting of a neurostimulation system according to the disclosure. This example begins with a patient being provided with an implantable stimulator configured to deliver electrical stimulation to at least one nerve (e.g., a hypoglossal nerve or one or more branches thereof), via a plurality of electrodes (step 301). The patient is further provided with an implantable controller for the stimulator, which is configured to control one or more parameters of the delivery of the electrical stimulation (e.g., a pulse amplitude, timing, duty cycle, etc.) (step 302). An electronic device (e.g., a dedicated controller operated by a clinician programmer) may then receive information identifying one or more covered and/or uncovered electrodes or pairs/combinations thereof within the plurality of electrodes or pairs available to the IPG (step 303). A user may utilize the electronic device to then transmit an instruction to the implantable controller for the stimulator to enter a first fitting mode (step 304). In this first fitting mode, the controller may then proceed to deliver stimulation, e.g., to one or more uncovered electrodes or pairs thereof (step 305) and to repeat the delivery step one or more times, gradually increasing the amplitude of stimulation applied in each cycle (step 306). As explained above in the context of FIG. 2, the initial pulse amplitude may be predetermined, a default value, or manually entered, and the parameter for increasing the amplitude is also subject to customization (e.g., it may be a fixed increment, or one subject to an increasing or decreasing rate of change, predetermined, manually entered, etc.). In any event, stimulation may be paused or otherwise ceased when the pulse amplitude reaches a predetermined maximum amount or upon user input (step 307), and the maximum pulse amplitude for each of the tested electrodes or pairs thereof may be recorded (step 308). The system may then optionally pause or otherwise wait for user input (step 309), or alternatively be programmed to automatically proceed according to a predetermined workflow (e.g., proceeding to one of the other fitting modes described herein). While this example illustrates the testing of electrodes or pairs thereof, it is envisioned that any combination of electrodes may be tested. Furthermore, as explained above, the systems described herein may also be configured to provide an interface allowing a user to select combinations of electrodes for testing based on a proximity or adjacency to a given electrode (or combination thereof). For example, the workflow shown in this example, or described in any other method herein, may further include a step of receiving user input identifying a parameter for determining adjacent or proximal electrodes and including any such electrode(s) which satisfy the given criteria in the testing process.

In it envisioned that the systems and methods described herein may be used to treat OSA or any other disease or condition for which neurostimulation is effective (e.g., the present methods may be used as a general platform for determining the optimal electrode(s), or combinations/pairs thereof to use for treatment, as well as the optimal pulse amplitude for each such electrode or combination/pair thereof. Thus, in still further aspects, the disclosure provides methods of treating a disease or condition in a human subject in need thereof, by delivering neurostimulation using a system configured using any of the methods described herein. In some aspects, the disease or condition may comprise OSA, depression, epilepsy (e.g., epileptic seizure frequency), and/or inflammation (e.g., related to maladies such as rheumatoid arthritis), brain diseases (e.g., Alzheimer's disease, dementia, traumatic brain injury, Parkinson's disease, and ischemic stroke), heart conditions (e.g., heart failure, cardiovascular disease, and for improving heart function, pain-related conditions (e.g., Migraines, cluster headaches, and pain-related disorders), inflammatory conditions (e.g., inflammatory bowel disease, rheumatoid arthritis, and autoimmune diseases, psychiatric conditions (e.g., anxiety disorders, and PTSD), and other conditions (e.g., diabetes, obesity, and sleep disorders). An exemplary method of treating any such diseases or conditions may comprise fitting a subject with a neurostimulation system according to the present disclosure and/or using any of the present systems to reduce one or more symptoms of the foregoing diseases and conditions.

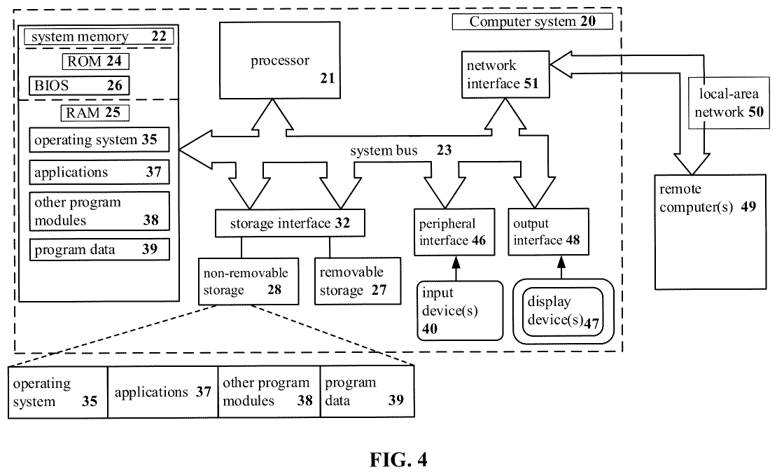

Aspects of the present disclosure may be implemented using hardware, software, or a combination thereof and may be implemented in one or more computer systems or other processing systems. In an aspect of the present disclosure, features are directed toward one or more computer systems capable of carrying out the functionality described herein. FIG. 4 is a block diagram illustrating an example of a computer system 20 which may be used to implement aspects of the systems and methods described herein. The computer system 20 can be in the form of multiple computing devices, or in the form of a single computing device, for example, a desktop computer, a notebook computer, a laptop computer, a mobile computing device, a smart phone, a tablet computer, a server, a mainframe, an embedded device, and other forms of computing devices.

As shown, the computer system 20 includes a central processing unit (CPU) 21, a system memory 22, and a system bus 23 connecting the various system components, including the memory associated with the central processing unit 21. The system bus 23 may comprise a bus memory or bus memory controller, a peripheral bus, and a local bus that is able to interact with any other bus architecture. Examples of the buses may include PCI, ISA, PCI-Express, HyperTransport™, InfiniBand™, Serial ATA, I2C, and other suitable interconnects. The central processing unit 21 (also referred to as a processor) can include a single or multiple sets of processors having single or multiple cores. The processor 21 may execute one or more computer-executable code implementing the techniques of the present disclosure. For example, any of commands/steps discussed in this specification, or shown in the accompanying drawings, may be performed by processor 21. The system memory 22 may be any memory for storing data used herein and/or computer programs that are executable by the processor 21. The system memory 22 may include volatile memory such as a random access memory (RAM) 25 and non-volatile memory such as a read only memory (ROM) 24, flash memory, etc., or any combination thereof. The basic input/output system (BIOS) 26 may store the basic procedures for transfer of information between elements of the computer system 20, such as those at the time of loading the operating system with the use of the ROM 24.

The computer system 20 may include one or more storage devices such as one or more removable storage devices 27, one or more non-removable storage devices 28, or a combination thereof. The one or more removable storage devices 27 and non-removable storage devices 28 are connected to the system bus 23 via a storage interface 32. In an aspect, the storage devices and the corresponding computer-readable storage media are power-independent modules for the storage of computer instructions, data structures, program modules, and other data of the computer system 20. The system memory 22, removable storage devices 27, and non-removable storage devices 28 may use a variety of computer-readable storage media. Examples of computer-readable storage media include machine memory such as cache, SRAM, DRAM, zero capacitor RAM, twin transistor RAM, eDRAM, EDO RAM, DDR RAM, EEPROM, NRAM, RRAM, SONOS, PRAM; flash memory or other memory technology such as in solid state drives (SSDs) or flash drives; magnetic cassettes, magnetic tape, and magnetic disk storage such as in hard disk drives or floppy disks; optical storage such as in compact disks (CD-ROM) or digital versatile disks (DVDs); and any other medium which may be used to store the desired data and which can be accessed by the computer system 20.

The system memory 22, removable storage devices 27, and non-removable storage devices 28 of the computer system 20 may be used to store an operating system 35, additional program applications 37, other program modules 38, and program data 39. The computer system 20 may include a peripheral interface 46 for communicating data from input devices 40, such as a keyboard, mouse, stylus, game controller, voice input device, touch input device, or other peripheral devices, such as a printer or scanner via one or more I/O ports, such as a serial port, a parallel port, a universal serial bus (USB), or other peripheral interface. A display device 47 such as one or more monitors, projectors, or integrated display, may also be connected to the system bus 23 across an output interface 48, such as a video adapter. In addition to the display devices 47, the computer system 20 may be equipped with other peripheral output devices (not shown), such as loudspeakers and other audiovisual devices.

The computer system 20 may operate in a network environment, using a network connection to one or more remote computers 49. The remote computer (or computers) 49 may be local computer workstations or servers comprising most or all of the aforementioned elements in describing the nature of a computer system 20. Other devices may also be present in the computer network, such as, but not limited to, routers, network stations, peer devices or other network nodes. The computer system 20 may include one or more network interfaces 51 or network adapters for communicating with the remote computers 49 via one or more networks such as a local-area computer network (LAN) 50, a wide-area computer network (WAN), an intranet, and the Internet. Examples of the network interface 51 may include an Ethernet interface, a Frame Relay interface, SONET interface, and wireless interfaces.

Aspects of the present disclosure may be a system, a method, and/or a computer program product. The computer program product may include a computer readable storage medium (or media) having computer readable program instructions thereon for causing a processor to carry out aspects of the present disclosure.

The computer readable storage medium can be a tangible device that can retain and store program code in the form of instructions or data structures that can be accessed by a processor of a computing device, such as the computing system 20. The computer readable storage medium may be an electronic storage device, a magnetic storage device, an optical storage device, an electromagnetic storage device, a semiconductor storage device, or any suitable combination thereof. By way of example, such computer-readable storage medium can comprise a random access memory (RAM), a read-only memory (ROM), EEPROM, a portable compact disc read-only memory (CD-ROM), a digital versatile disk (DVD), flash memory, a hard disk, a portable computer diskette, a memory stick, a floppy disk, or even a mechanically encoded device such as punch-cards or raised structures in a groove having instructions recorded thereon. As used herein, a computer readable storage medium is not to be construed as being transitory signals per se, such as radio waves or other freely propagating electromagnetic waves, electromagnetic waves propagating through a waveguide or transmission media, or electrical signals transmitted through a wire.

Computer readable program instructions described herein can be downloaded to respective computing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network interface in each computing device receives computer readable program instructions from the network and forwards the computer readable program instructions for storage in a computer readable storage medium within the respective computing device.

Computer readable program instructions for carrying out operations of the present disclosure may be assembly instructions, instruction-set-architecture (ISA) instructions, machine instructions, machine dependent instructions, microcode, firmware instructions, state-setting data, or either source code or object code written in any combination of one or more programming languages, including an object-oriented programming language, and conventional procedural programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a LAN or WAN, or the connection may be made to an external computer (for example, through the Internet). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present disclosure.

In various aspects, the systems and methods described in the present disclosure can be addressed in terms of modules. The term “module” as used herein refers to a real-world device, component, or arrangement of components implemented using hardware, such as by an application specific integrated circuit (ASIC) or FPGA, for example, or as a combination of hardware and software, such as by a microprocessor system and a set of instructions to implement the module's functionality, which (while being executed) transform the microprocessor system into a special-purpose device. A module may also be implemented as a combination of the two, with certain functions facilitated by hardware alone, and other functions facilitated by a combination of hardware and software. In certain implementations, at least a portion, and in some cases, all, of a module may be executed on the processor of a computer system. Accordingly, each module may be realized in a variety of suitable configurations, and should not be limited to any particular implementation exemplified herein.

In the interest of clarity, not all of the routine features of the aspects are disclosed herein. It would be appreciated that in the development of any actual implementation of the present disclosure, numerous implementation-specific decisions must be made in order to achieve the developer's specific goals, and these specific goals will vary for different implementations and different developers. It is understood that such a development effort might be complex and time-consuming, but would nevertheless be a routine undertaking of engineering for those of ordinary skill in the art, having the benefit of this disclosure.

Furthermore, it is to be understood that the phraseology or terminology used herein is for the purpose of description and not of restriction, such that the terminology or phraseology of the present specification is to be interpreted by the skilled in the art in light of the teachings and guidance presented herein, in combination with the knowledge of those skilled in the relevant art(s). Moreover, it is not intended for any term in the specification or claims to be ascribed an uncommon or special meaning unless explicitly set forth as such.

The various aspects disclosed herein encompass present and future known equivalents to the known modules referred to herein by way of illustration. Moreover, while aspects and applications have been shown and described, it would be apparent to those skilled in the art having the benefit of this disclosure that many more modifications than mentioned above are possible without departing from the inventive concepts disclosed herein.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular compound, composition, article, apparatus, methodology, protocol, and/or reagent, etc., described herein, unless expressly stated as such. In addition, those of ordinary skill in the art will recognize that certain changes, modifications, permutations, alterations, additions, subtractions and sub-combinations thereof can be made in accordance with the teachings herein without departing from the spirit of the present specification. It is therefore intended that the following appended claims and claims hereafter introduced are interpreted to include all such changes, modifications, permutations, alterations, additions, subtractions and sub-combinations as are within their true spirit and scope.

Certain aspects of the present disclosure are described herein, including the best mode known to the inventors for carrying out the disclosure. Of course, variations on these described embodiments will become apparent to those of ordinary skill in the art upon reading the foregoing description. The inventor expects skilled artisans to employ such variations as appropriate, and the inventors intend for the present disclosure to be practiced otherwise than specifically described herein. Accordingly, this disclosure includes all modifications and equivalents of the subject matter recited in the claims appended hereto as permitted by applicable law. Moreover, any combination of the above-described embodiments in all possible variations thereof is encompassed by the disclosure unless otherwise indicated herein or otherwise clearly contradicted by context.

Groupings of alternative embodiments, elements, or steps of the present disclosure are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques.

Use of the terms “may” or “can” in reference to an embodiment or aspect of an embodiment also carries with it the alternative meaning of “may not” or “cannot.” As such, if the present specification discloses that an embodiment or an aspect of an embodiment may be or can be included as part of the inventive subject matter, then the negative limitation or exclusionary proviso is also explicitly meant, meaning that an embodiment or an aspect of an embodiment may not be or cannot be included as part of the inventive subject matter. In a similar manner, use of the term “optionally” in reference to an embodiment or aspect of an embodiment means that such embodiment or aspect of the embodiment may be included as part of the inventive subject matter or may not be included as part of the inventive subject matter. Whether such a negative limitation or exclusionary proviso applies will be based on whether the negative limitation or exclusionary proviso is recited in the claimed subject matter.

Notwithstanding that the numerical ranges and values setting forth the broad scope of the disclosure are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

The terms “a,” “an,” “the” and similar references used in the context of describing aspects of the present disclosure (especially in the context of the following claims) are to be construed to cover both the singular and the plural, unless otherwise indicated herein or clearly contradicted by context. Further, ordinal indicators—such as “first,” “second,” “third,” etc.—for identified elements are used to distinguish between the elements, and do not indicate or imply a required or limited number of such elements, and do not indicate a particular position or order of such elements unless otherwise specifically stated. All methods described herein can be performed in any suitable order unless otherwise indicated herein or otherwise clearly contradicted by context. The use of any and all examples, or “exemplary” language (e.g., “such as”) provided herein is intended merely to better illuminate the present disclosure and does not pose a limitation on the scope of the invention otherwise claimed. No language in the present specification should be construed as indicating any non-claimed element essential to the practice of the invention.

When used in the claims, whether as filed or added per amendment, the open-ended transitional term “comprising” (and equivalent open-ended transitional phrases thereof like including, containing and having) encompasses all the expressly recited elements, limitations, steps and/or features alone or in combination with unrecited subject matter; the named elements, limitations and/or features are essential, but other unnamed elements, limitations and/or features may be added and still form a construct within the scope of the claim. Specific embodiments disclosed herein may be further limited in the claims using the closed-ended transitional phrases “consisting of” or “consisting essentially of” in lieu of or as an amended for “comprising.” When used in the claims, whether as filed or added per amendment, the closed-ended transitional phrase “consisting of” excludes any element, limitation, step, or feature not expressly recited in the claims. The closed-ended transitional phrase “consisting essentially of” limits the scope of a claim to the expressly recited elements, limitations, steps and/or features and any other elements, limitations, steps and/or features that do not materially affect the basic and novel characteristic(s) of the claimed subject matter. Thus, the meaning of the open-ended transitional phrase “comprising” is being defined as encompassing all the specifically recited elements, limitations, steps and/or features as well as any optional, additional unspecified ones. The meaning of the closed-ended transitional phrase “consisting of” is being defined as only including those elements, limitations, steps and/or features specifically recited in the claim whereas the meaning of the closed-ended transitional phrase “consisting essentially of” is being defined as only including those elements, limitations, steps and/or features specifically recited in the claim and those elements, limitations, steps and/or features that do not materially affect the basic and novel characteristic(s) of the claimed subject matter. Therefore, the open-ended transitional phrase “comprising” (and equivalent open-ended transitional phrases thereof) includes within its meaning, as a limiting case, claimed subject matter specified by the closed-ended transitional phrases “consisting of” or “consisting essentially of.” As such embodiments described herein or so claimed with the phrase “comprising” are expressly or inherently unambiguously described, enabled and supported herein for the phrases “consisting essentially of” and “consisting of.”

All patents, patent publications, and other publications referenced and identified in the present specification are individually and expressly incorporated herein by reference in their entirety for the purpose of describing and disclosing, for example, the compositions and methodologies described in such publications that might be used in connection with the present disclosure. These publications are provided solely for their disclosure prior to the filing date of the present application. Nothing in this regard should be construed as an admission that the inventors are not entitled to antedate such disclosure by virtue of prior disclosure or for any other reason. All statements as to the date or representation as to the contents of these documents is based on the information available to the applicants and does not constitute any admission as to the correctness of the dates or contents of these documents.

Lastly, the terminology used herein is for the purpose of describing particular embodiments only, and is not intended to limit the scope of the present invention, which is defined solely by the claims. Accordingly, the present invention is not limited to that precisely as shown and described.

1. A neurostimulation system, comprising:
an implantable stimulator configured to deliver electrical stimulation to at least one nerve of a human subject via a plurality of electrodes or electrode pairs, and
an implantable controller configured to control one or more parameters of the delivery of the electrical stimulation to the at least one nerve of the human subject,
wherein the controller is configured to operate in a first fitting mode, based on user input wirelessly communicated to the controller, the first fitting mode comprising a mode wherein the controller is configured to:
a) deliver stimulation sequentially, for a predetermined stimulation duration and at a predetermined pulse amplitude, to each of at least a subset of the plurality of electrodes or electrode pairs,
b) repeat step a) one or more times, gradually increasing a pulse amplitude of stimulation during each repeat cycle, starting from the predetermined pulse amplitude,
c) cease delivering stimulation, when a predetermined maximum pulse amplitude is reached or upon receipt of user input instructing the controller to cease stimulation, and
d) store a pulse amplitude parameter for one or more of the electrodes or electrode pairs used in steps a)-c), and optionally a selection of one or more of the electrodes or electrode pairs used in steps a)-c), provided via user input, wherein the stored parameter is indicative of a minimum threshold for stimulation.

2. The system of claim 1, wherein the controller is further configured to operate in a second fitting mode, based on user input wirelessly communicated to the controller from an electronic device, the second fitting mode comprising a mode wherein the controller is configured to:
a) deliver stimulation to at least one electrode or electrode pair selected based on user input, for a predetermined stimulation duration and at a predetermined pulse amplitude,
b) gradually increase a pulse amplitude of stimulation delivered to the electrode or electrode pair selected in step a), starting from the predetermined pulse amplitude,
c) stop delivering stimulation, when a predetermined maximum pulse amplitude is reached or upon receipt of user input instructing the controller to cease stimulation, and
d) store a pulse amplitude parameter for one or more of the electrodes or electrode pairs used in steps a)-c), wherein the stored parameter is indicative of a maximum threshold for stimulation.

3. The system of claim 1, wherein the controller is further configured to operate in a third fitting mode, based on user input wirelessly communicated to the controller from an electronic device, the third fitting mode comprising a mode wherein the controller is configured to:
a) receive minimum and maximum pulse amplitude parameters for at least one electrode or electrode pair in the plurality of electrodes or electrode pairs, based on user input wirelessly communicated to the controller from the electronic device.

4. The system of claim 1, wherein the electronic device comprises a dedicated controller of the neurostimulation system, or a smartphone, tablet, or computer configured to execute a software application that provides communication with and control of the controller.

5. The system of claim 1, wherein the predetermined stimulation duration used in one or more of the fitting modes, comprises a) 0.5, 1.0, 1.5, 2.0, 2.5, 3.0, 3.5, 4.0, 4.5, or 5.0 seconds, or b) a duration within a range defined by a pair of endpoints selected from any of the lengths of time listed in a).

6. The system of claim 1, wherein the predetermined pulse amplitude used in one or more of the fitting modes, comprises a) 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, 0.25, 0.30, 0.35, 0.40, 0.45, 0.50, 0.60, 0.70, 0.80, 0.90, 1.00, 1.10, 1.20, 1.30, 1.40, 1.50, 1.60, 1.70, 1.80, 1.90, 2.00, 2.50, 3.00, 3.50, 4.00, 4.50, 5.00, 5.50, or 6.00 mA, or b) a pulse amplitude within a range defined by a pair of endpoints selected from any of the amounts listed in a).

7. The system of claim 1, wherein the controller is configured to gradually increase the pulse amplitude delivered in step b), in one or more of the fitting modes, by incrementing the pulse amplitude by i) 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, 0.25, 0.30, 0.35, 0.40, 0.45, 0.50, 0.60, 0.70, 0.80, 0.90, 1.00, 1.10, 1.20, 1.30, 1.40, 1.50, 1.60, 1.70, 1.80, 1.90, 2.00, 2.50, 3.00, 3.50, 4.00, 4.50, 5.00, 5.50, or 6.00 mA each time, or ii) by incrementing the pulse amplitude by an amount within a range defined by a pair of endpoints selected from any of the amounts listed in i).

8. The system of claim 3, wherein the controller is configured to operate in the first fitting mode, the second fitting mode, and the third fitting mode, and alternate between, activate, or deactivate, a current fitting mode, based on user input received from the electronic device.

9. A method for fitting a human subject with a neurostimulation system, comprising:
providing an implantable stimulator configured to deliver electrical stimulation to at least one nerve of a human subject via a plurality of electrodes or electrode pairs;
providing an implantable controller configured to control one or more parameters of the delivery of the electrical stimulation to the at least one nerve of the human subject;
receiving, by an electronic device configured to wirelessly communicate with and control one or more parameters of the controller, information identifying one or more covered and/or uncovered electrodes or electrode pairs in the plurality of electrodes or electrode pairs;
transmitting, by the electronic device, an instruction to the controller to initiate a first fitting mode, the first fitting mode comprising a mode wherein the controller is configured to:
a) deliver stimulation sequentially, for a predetermined stimulation duration and at a predetermined pulse amplitude, to each of the uncovered electrodes or electrode pairs,
b) repeat step a) one or more times, gradually increasing a pulse amplitude of stimulation during each repeat cycle, starting from the predetermined pulse amplitude,
c) cease delivering stimulation, when a predetermined maximum pulse amplitude is reached or upon receipt of user input instructing the controller to cease stimulation, and
d) store a pulse amplitude parameter for one or more of the uncovered electrodes or electrode pairs used in steps a)-c), wherein the stored parameter is indicative of a minimum threshold for stimulation.

10. The method of claim 9, wherein the controller is further configured to operate in a second fitting mode, the second fitting mode comprising a mode wherein the controller is configured to:
a) deliver stimulation to at least one electrode or electrode pair selected based on user input, for a predetermined stimulation duration and at a predetermined pulse amplitude,
b) gradually increase a pulse amplitude of stimulation delivered to the electrode or electrode pair selected in step a), starting from the predetermined pulse amplitude,
c) stop delivering stimulation, when a predetermined maximum pulse amplitude is reached or upon receipt of user input instructing the controller to cease stimulation, and
d) store a pulse amplitude parameter for one or more of the electrodes or electrode pairs used in steps a)-c), wherein the stored parameter is indicative of a maximum threshold for stimulation; and the method further comprises
transmitting, by the electronic device, an instruction to the controller to initiate the second fitting mode.

11. The method of claim 9, wherein the controller is further configured to operate in a third fitting mode, the third fitting mode comprising a mode wherein the controller is configured to: a) receive minimum and maximum pulse amplitude parameters for at least one electrode or electrode pair in the plurality of electrodes or electrode pairs; and the method further comprises
transmitting, by the electronic device, an instruction to the controller to initiate the third fitting mode.

12. The method of claim 9, wherein the electronic device comprises a dedicated controller of the neurostimulation system, or a smartphone, tablet, or computer configured to execute a software application that provides communication with and control of the controller.

13. The method of claim 9, wherein the predetermined stimulation duration used in one or more of the fitting modes, comprises a) 0.5, 1.0, 1.5, 2.0, 2.5, 3.0, 3.5, 4.0, 4.5, or 5.0 seconds, or b) a duration within a range defined by a pair of endpoints selected from any of the lengths of time listed in a).

14. The method of claim 9, wherein the predetermined pulse amplitude used in one or more of the fitting modes, comprises a) 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, 0.25, 0.30, 0.35, 0.40, 0.45, 0.50, 0.60, 0.70, 0.80, 0.90, 1.00, 1.10, 1.20, 1.30, 1.40, 1.50, 1.60, 1.70, 1.80, 1.90, 2.00, 2.50, 3.00, 3.50, 4.00, 4.50, 5.00, 5.50, or 6.00 mA, or b) a pulse amplitude within a range defined by a pair of endpoints selected from any of the amounts listed in a).

15. The method of claim 9, wherein the controller is configured to gradually increase the pulse amplitude delivered in step b), in one or more of the fitting modes, by incrementing the pulse amplitude by i) 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, 0.25, 0.30, 0.35, 0.40, 0.45, 0.50, 0.60, 0.70, 0.80, 0.90, 1.00, 1.10, 1.20, 1.30, 1.40, 1.50, 1.60, 1.70, 1.80, 1.90, 2.00, 2.50, 3.00, 3.50, 4.00, 4.50, 5.00, 5.50, or 6.00 mA each time, or ii) by incrementing the pulse amplitude by an amount within a range defined by a pair of endpoints selected from any of the amounts listed in i).

16. The method of claim 11, further comprising:
transmitting, by the electronic device to the controller, instructions to initiate the first fitting mode, followed by instructions to enter the second fitting mode, and/or the third fitting mode.

17. The method of claim 9, wherein the controller is further configured to pause between any or all of steps a)-d) of the first fitting mode and/or the second fitting mode, until an instruction to proceed is received from the electronic device.

18. The method of claim 9, wherein the instruction transmitted by the electronic device comprises user input provided by a clinician programmer.

19. The method of claim 9, wherein the electronic device is a dedicated controller comprising a portable electronic device configured to wirelessly communicate with and control a plurality of neurostimulation systems.

20. A method for adjusting or optimizing a neurostimulation system, comprising:
providing an implantable stimulator configured to deliver electrical stimulation to at least one nerve of a human subject via a plurality of electrodes or electrode pairs;
providing an implantable controller configured to control one or more parameters of the delivery of the electrical stimulation to the at least one nerve of the human subject;
receiving, by an electronic device configured to wirelessly communicate with and control one or more parameters of the controller, information identifying one or more electrodes or electrode pairs in the plurality of electrodes or electrode pairs;
transmitting, by the electronic device, an instruction to the controller to initiate a first fitting mode, the first fitting mode comprising a mode wherein the controller is configured to:
a) deliver stimulation sequentially, for a predetermined stimulation duration and at a predetermined pulse amplitude, to one or more electrodes or electrode pairs positioned adjacent to at least one of the identified electrodes or electrode pairs,
b) repeat step a) one or more times, gradually increasing a pulse amplitude of stimulation during each repeat cycle, starting from the predetermined pulse amplitude,
c) cease delivering stimulation, when a predetermined maximum pulse amplitude is reached or upon receipt of user input instructing the controller to cease stimulation, and
d) store a pulse amplitude parameter for one or more of the electrodes or electrode pairs stimulated in steps a)-c), wherein the stored parameter is indicative of a minimum threshold for stimulation, and optionally a selection of one or more of the electrodes or electrode pairs stimulated in steps a)-c), received from the electronic device.
